(19) United States
(12) Patent Application Publication
(10) Pub. No.: US 2023/0381067 A1
(43) Pub. Date: Nov. 30, 2023

(54) PROCEDURE FOR TEMPORARILY PLACING CUSTOM TOOTH-IMAGES ON TEETH THAT ARE REMOVABLE BY A DENTAL PROFESSIONAL

(71) Applicant: Scott Aaron Smith, Astoria, OR (US)

(72) Inventor: Scott Aaron Smith, Astoria, OR (US)

(21) Appl. No.: 18/307,348

(22) Filed: Apr. 26, 2023

Related U.S. Application Data
(60) Provisional application No. 63/363,898, filed on Apr. 29, 2022.
(60) Provisional application No. 63/373,270, filed on Aug. 23, 2022.

(51) Int. Cl. A61K 6/30; A61K 6/78; A61K 6/838; A61C 13/15

(52) U.S. Cl. CPC A61K 6/30; A61K 6/78; A61K 6/838; A61C 19/003

(57) ABSTRACT

A method and system for applying colored dental adhesive and a high-resolution decal to the surface of a person's tooth. The colored dental adhesive is formed by combining dental adhesive with a food grade colorant. The high-resolution decal is formed by printing a design on a paper substrate. Before application of the colored dental adhesive, the tooth is cleaned and etched with a phosphoric acid compound. The colored dental adhesive is applied to the surface of a tooth. The decal is then placed on colored dental adhesive, and the colored dental adhesive is cured. Additional layers of dental adhesive may be applied on top of the decal.

### INCORPORATION BY REFERENCE TO ANY PRIORITY APPLICATIONS

This application claims priority to U.S. Provisional Patent Application No. 63/363,898, filed Apr. 29, 2022, and U.S. Provisional Patent Application No. 63/373,270 filed Aug. 23, 2022, the entirety of each of which are incorporated herein by reference. Any and all applications for which a foreign or domestic priority claim is identified in the Application Data Sheet as filed with the present application are hereby incorporated by reference under 37 CFR 1.57.

### TECHNICAL FIELD

Embodiments of the present application relate to a method and kit for applying decals to teeth. More particularly, the embodiments of the present application relate to a method for applying colored dental adhesive to the surface of a tooth and applying a high-resolution decal to the colored tooth.

### BACKGROUND

Many people like to differentiate themselves from others by modifying their body. In some instances, people differentiate themselves with permanent and non-permanent tattoos. Many body parts can be tattooed or colored permanently, or for an extended period of time, with intricate high-resolution designs.

### SUMMARY

Embodiments of the present application provide a method and system for applying a colored dental adhesive and a decal with a high resolution design to the surface of a tooth.

In some configurations, a method of applying an esthetic alteration on a tooth surface can include the steps of: applying a first layer of dental adhesive to the tooth surface; placing a decal on the first layer of dental adhesive; applying at least one additional layer of dental adhesive on top of the decal; and curing the at least one additional layer of dental adhesive.

In some configurations, the method can include the step of curing the first layer of dental adhesive after applying the first layer of dental adhesive.

In some configurations, the first layer of dental adhesive can be a colored dental adhesive including a pigment.

In some configurations, the pigment can include at least one of pearlescent mica, matte mica, titanium dioxide, zinc oxide, iron oxide, aluminum oxide, or chromium-cobalt-aluminum oxide.

In some configurations, each layer of dental adhesive can be an eighth-generation dental adhesive.

In some configurations, the tooth surface can be etched with a 38% phosphoric acid compound.

In some configurations, the decal can include a design printed on a paper substrate.

In some configurations, the paper substrate can include rice paper or tissue paper, and the paper substrate can include a thickness between 8 microns and 10 microns.

In some configurations, the method can include the step of altering the layers of dental adhesive with a dental bur.

In some configurations, the method can include the step of removing, by a dental professional, the esthetic alteration.

In some configurations, the esthetic alteration can be removable with a high-speed dental handpiece.

In some configurations, the esthetic alteration can be removable with a slow-speed dental handpiece.

In some configurations, an esthetic alteration kit for a tooth can include: at least one dental adhesive; at least one pigment; and at least one high resolution decal printed on a paper substrate.

In some configurations, the at least one high resolution decal can include a resolution of at least 4800×1200 dots per inch.

In some configurations, the pigment can include at least one of, pearlescent mica, matte mica, titanium dioxide, zinc oxide, iron oxide, aluminum oxide, or chromium-cobalt-aluminum oxide.

In some configurations, a method of constructing an esthetic alteration kit for a tooth can include the steps of: combining a dental adhesive with a pigment to form a colored dental adhesive; and printing a design on a paper substrate to form a decal.

In some configurations, the pigment can include at least one of pearlescent mica, matte mica, titanium dioxide, zinc oxide, iron oxide, aluminum oxide, or chromium-cobalt-aluminum oxide.

In some configurations, the paper substrate can include rice paper or tissue paper.

In some configurations, the paper substrate can include a thickness between 8 microns and 10 microns.

In some configurations, the design can include a minimum resolution of 4800×1200 dots per inch.

In some configurations, an esthetic alteration kit for a tooth can include: a dental adhesive; an applicator; an adhesive curing device; a tooth isolator; and at least one high resolution decal printed on a paper substrate.

In some configurations, the dental adhesive can be a unit dose of resin tooth bonding agent.

In some configurations, wherein the dental adhesive can be a colored dental adhesive.

In some configurations, the esthetic alteration kit can include one or more pigments and/or color additives, wherein the one or more pigments include at least one of, pearlescent mica, matte mica, titanium dioxide, zinc oxide, iron oxide, aluminum oxide, or chromium-cobalt-aluminum oxide, and wherein the one or more color additives includes Food, Drugs, and Cosmetics (FD&C) and/or Drugs & Cosmetics (D&C) color additives.

In some configurations, the at least one high resolution decal can include a resolution of at least 4800×1200 dots per inch.

In some configurations, the applicator can be a microbrush.

In some configurations, the adhesive curing device can be a curing light.

In some configurations, the tooth isolator can be one or more cotton rolls.

In some configurations, the method, can include the step of applying a resin overcoat on the at least one additional layer of dental adhesive.

In some configurations, the pigment can be a color additive including Food, Drugs and Cosmetics (FD&C), and/or Drugs & Cosmetics (D&C) color additives.

In some configurations, the decal can include a pigment based ink, and/or a dye based ink.

In some configurations, combining the dental adhesive with the pigment can be performed by a dentist, a user or a third party.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure:

FIG. 1 illustrates a schematic diagram of the steps in the method of an embodiment of the method of the present application.

FIG. 2 illustrates an example of a colored dental adhesive of the present application applied to a tooth.

FIG. 3 illustrates an example of a decal with a plurality of designs according to an embodiment of the present application.

FIG. 4 illustrates an example of a design of the present application applied to a tooth.

FIG. 5 illustrates an example of a kit of the present application.

FIG. 6 illustrates a schematic diagram of the steps in the method of an embodiment of the method of the present application

FIG. 7 illustrates an example of a second kit of the present application.

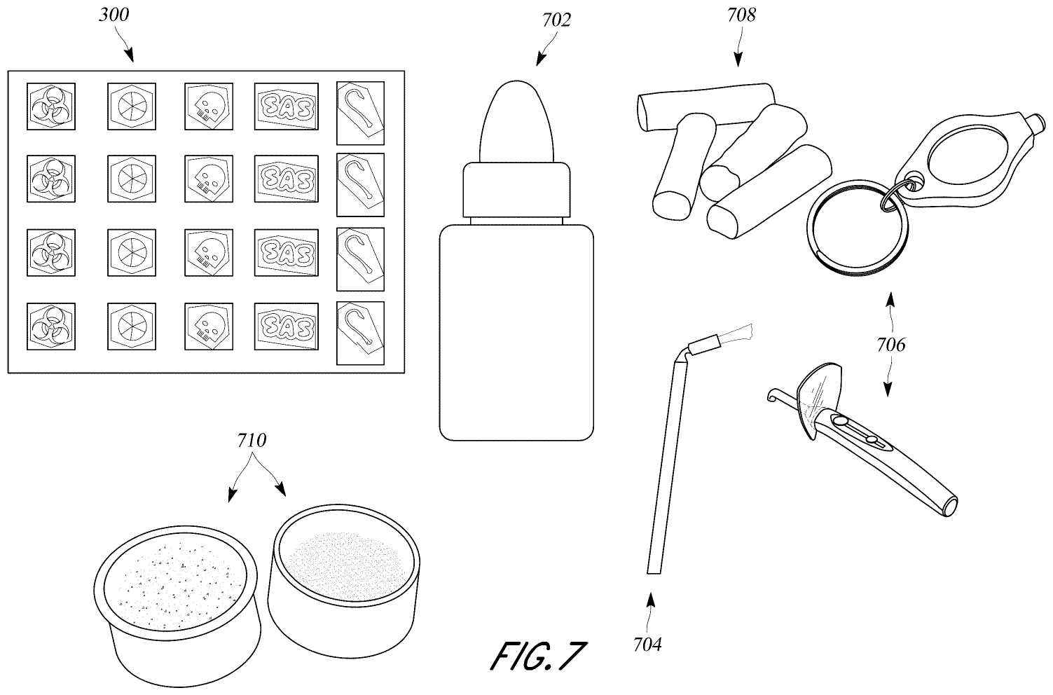

### DETAILED DESCRIPTION

These and other features, aspects, and advantages of the present disclosure are described with reference to the drawings of certain embodiments, which are intended to schematically illustrate certain embodiments and not to limit the disclosure.

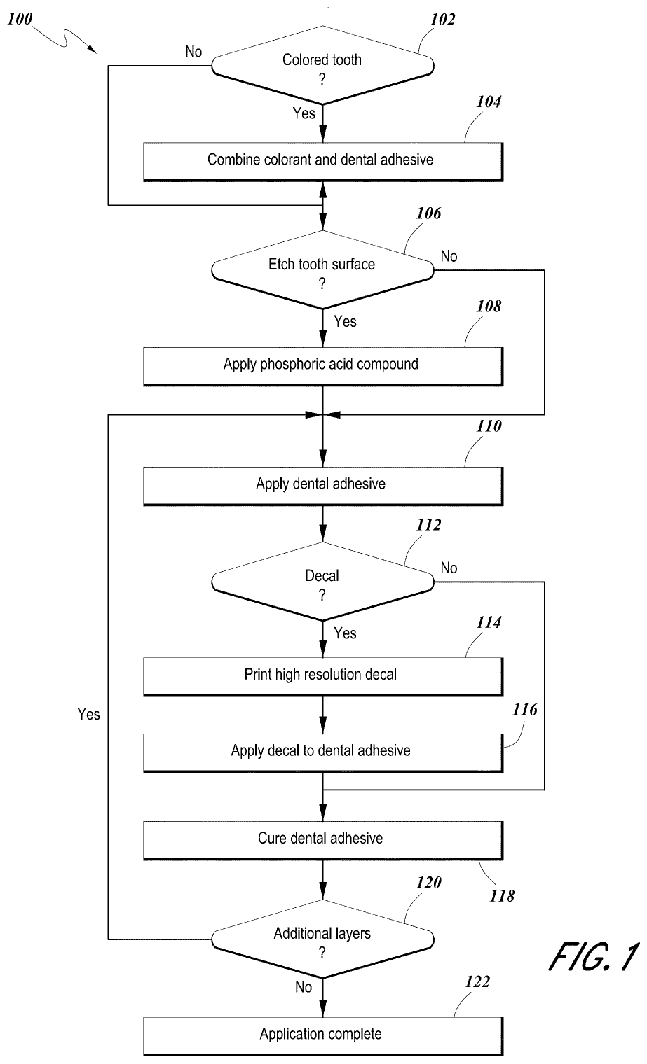

FIG. 1 illustrates a schematic diagram of the method 100 of applying an esthetic alteration to the surface of a patient's tooth. In some embodiments, some or all of steps 102-122 of method 100 may be performed by a dental professional, a patient, a third party or any combination of the three. The first step of the method of the illustrated embodiment is generally indicated by the numeral 102. Step 102 calls for choosing whether a surface of the tooth will be colored.

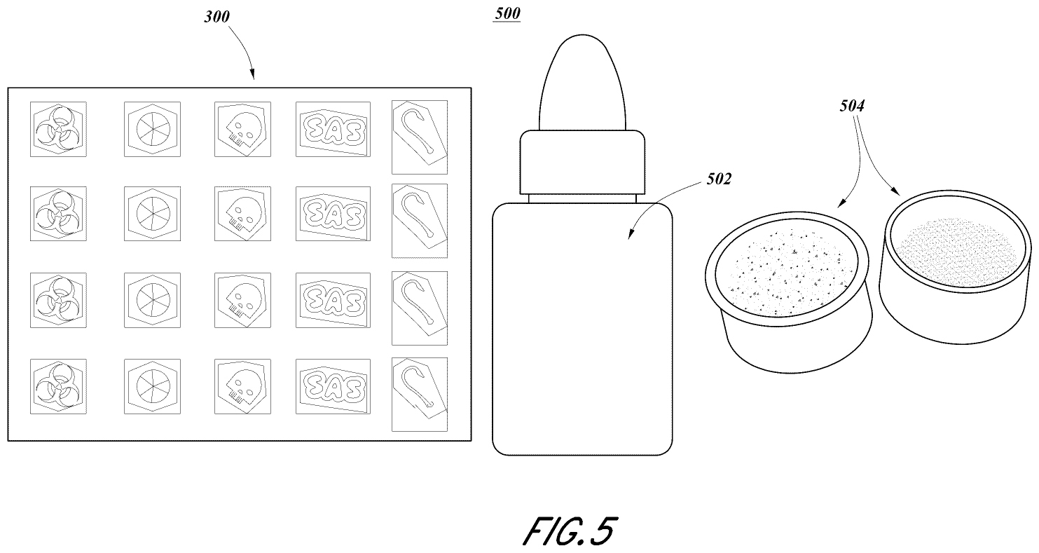

If the surface of the tooth will be colored, the next step is generally indicated by the numeral 104. Step 104 calls for combining a colorant or pigment, and a dental adhesive. In some embodiments, the colorant may include at least one of pearlescent mica, matte mica, titanium dioxide, zinc oxide, iron oxide, aluminum oxide, or chromium-cobalt-aluminum oxide, or any other non-toxic pigment. The dental adhesive may be any dental adhesive or bonding agent typically used by dental professionals. In some embodiments, the dental adhesive may include eighth-generation dental adhesive. In some embodiments, the colorant and the dental adhesive may be mixed by a third party, and the dental professional may receive the colorant and the dental adhesive premixed in a kit 500 as shown in FIG. 5.

If at step 102 the tooth will not be colored, step 104 may be skipped. In this instance, dental adhesive without colorant may be used to complete the remaining steps of the method 100.

The next step is generally indicated by the numeral 106. Step 106 calls for deciding if the surface of the tooth will be etched. If the surface of the tooth will be etched, the next step is generally indicated by the numeral 108. Step 108 calls for applying an acid compound to the surface of the tooth. In some embodiments, the acid compound may include one of 30 percent, 31 percent, 32 percent, 33 percent, 34 percent, 35 percent, 36 percent, 37 percent, 38 percent, 39 percent, or 40 percent, or any other percentage of acid. In some embodiments, the acid compound comprises between 30 and 40 percent acid. In some embodiments, the acid may comprise phosphoric acid, nitric acid, or any other acid commonly used for etching. In some embodiments, the acid may be applied with a cotton ball, cotton swab, roll of cotton, microbrush, or any other applicator commonly used for applying acid to the surface of a tooth.

If at step 106 the surface of the tooth will not be etched, step 108 may be skipped. In some embodiments, prior to step 108, the surface of the tooth may be cleaned with hydrogen peroxide, a toothbrush, pumice or other abrasive, a prophy cup, a prophy brush, a prophy jet, an air polisher, a sickle scaler and/or any other dental tool used for cleaning the surface of a tooth.

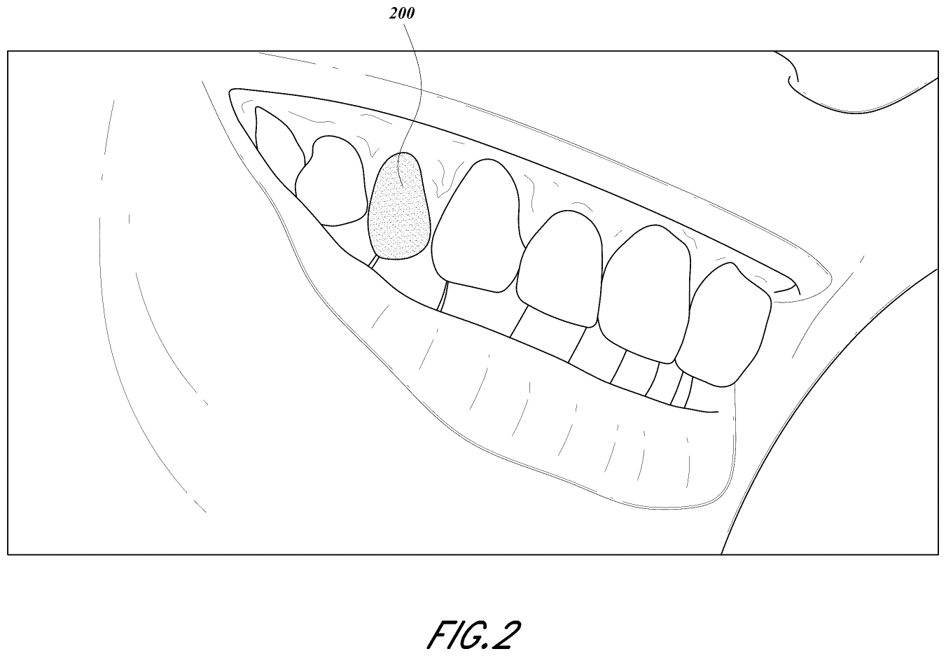

The next step is generally indicated by the numeral 110. Step 110 calls for applying the dental adhesive to the surface of the tooth. If at step 102, the tooth will be colored, the dental adhesive used at step 110 may be the colored dental adhesive. FIG. 2 shows an example of a tooth 200 with colored dental adhesive applied to a surface of the tooth 200. If at step 102 the tooth will not be colored and step 104 is skipped, the dental adhesive used at step 110 may be substantially clear dental adhesive. The dental adhesive may be applied at step 110 with a cotton ball, cotton swab, roll of cotton, microbrush, or any other applicator commonly used for applying dental adhesive to the surface of a tooth.

The next step is generally indicated by numeral 112. Step 112 calls for deciding if a design 302 will be applied on top of the dental adhesive on the surface of the tooth.

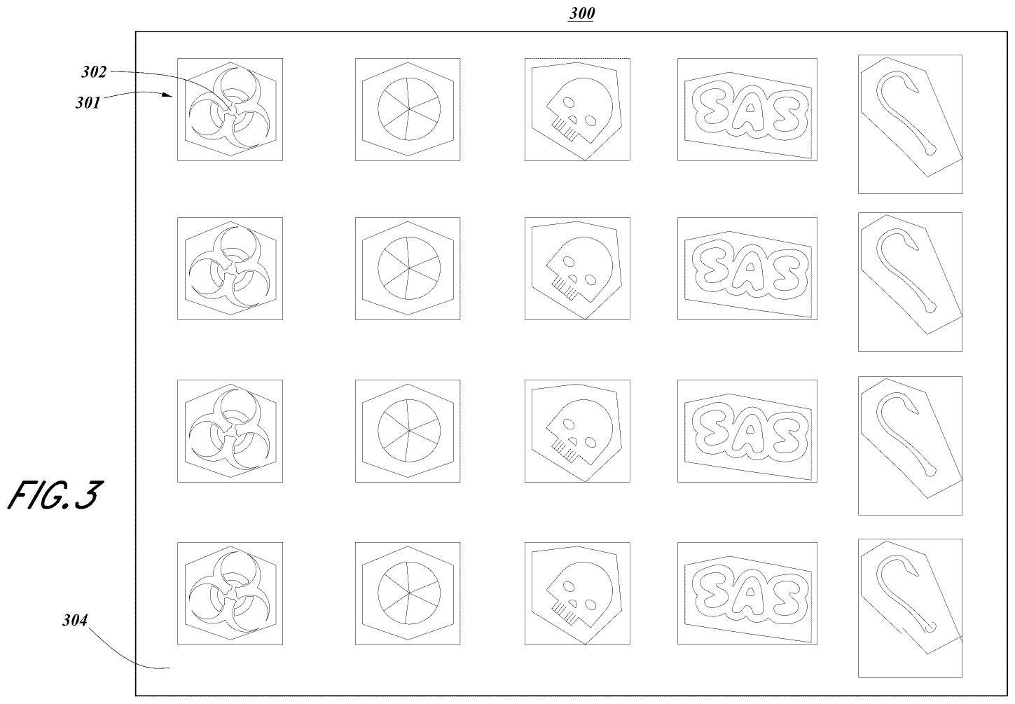

If at step 112, the design 302 will be applied to the surface of the tooth or the dental adhesive, the next step, generally indicated by the numeral 114, calls for printing a high-resolution decal 301. As shown in FIG. 3, the decal 301 may comprise a design 302 printed on a paper substrate 304. One or more designs 302 may be printed on the same paper substrate 304 forming a sheet of decals 300. The designs 302 may be printed with a high-resolution printer. In some embodiments, the printer, the design 302 and/or the decal 300 may comprise a minimum resolution of 4800×1200 dots per inch (DPI). In some embodiments, the printer, the design 302 and/or the decal 301 may comprise a resolution less than 4800×1200 DPI. In some embodiments, the printer may comprise an inkjet printer or a laser printer. In some embodiments, the paper substrate 304 may comprise rice paper, tissue paper or any other paper substrate. In some embodiments, the paper substrate 304 may comprise a thickness. In some embodiments, the thickness may be 8 microns to 10 microns. In some embodiments, the thickness may be 1 micron to 100 microns. In some embodiments, step 114 is performed by a dental professional. In some embodiments, step 114 may be performed by a third party prior to step 102. In these embodiments, the sheet of decals 300 and a colored adhesive 502 may comprise a kit 500 as shown in FIG. 5. The kit 500 may be delivered to a dental professional with the designs 302 already printed on the paper substrate 304, and the adhesive already comprising a colored adhesive 502. In some embodiments, the kit 500 may comprise the sheet of decals 300 and one or more colorants or pigments 504. In some embodiments, the kits 500 may comprise the sheet of decals 300, an uncolored adhesive 502 and one or more colorants or pigments 504.

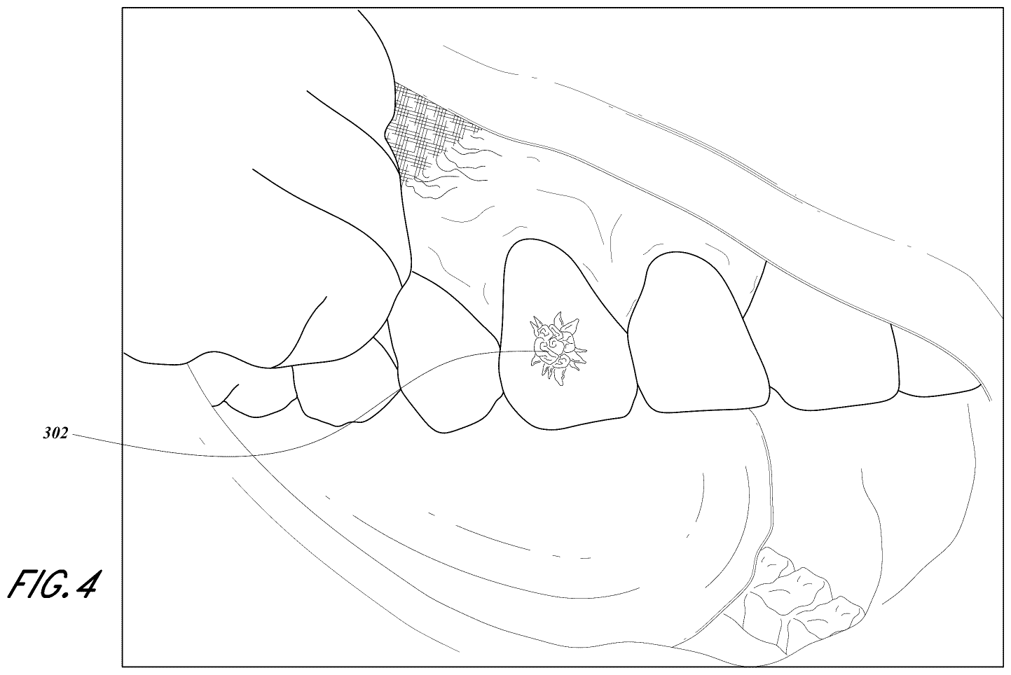

The next step is generally indicated by numeral 116. Step 116 calls for applying or placing the decal 301 on the surface of the tooth or dental adhesive. The decal 301 may be cut or otherwise separated from the sheet of decals 300 prior to applying the decal 301 to the surface of the tooth. The decal 301 may be applied with tweezers, or any other dental tool. FIG. 4 shows an example of a tooth with a decal 301 applied to a surface of the tooth. As shown in FIG. 4, in some embodiments, after the decal 301 is applied to the surface of the tooth, the paper substrate 304 may not be visible. In these embodiments, the design 302 may be visible on the surface of the tooth even though the paper substrate 304 is not visible.

If at step 112, a decal will not be applied to the surface of the tooth, steps 114 and 116 may be skipped.

The next step is generally indicated by numeral 118. Step 118 calls for curing the dental adhesive. In some embodiments, light and/or heat may be used to cure the dental adhesive. In some embodiments, the dental adhesive may be a self-curing adhesive.

The next step is generally indicated by numeral 120. Step 120 calls for deciding if additional layers of dental adhesive may be applied. If additional layers may be applied, the steps of the disclosed methods starting at step 110 may be repeated.

If additional layers may not be applied, the next step, generally indicated by numeral 122, is completing the application of the esthetic alteration of method 100.

In some embodiments, the method 100 may further include the step of adjusting a surface of the dental adhesive with high-speed dental burs, slow-speed dental burs or any other dental handpiece, after each layer of dental adhesive is applied. In some embodiments, the method 100 may further include the step of removing the esthetic alteration.

In some embodiments, the dental adhesive and/or the decal may stay on the surface for a period of time. In some embodiments the period of time may be 3-6 months. In some embodiments, the period of time may be 1-12 months.

It is to be appreciated that although the steps of method 100 are described in a particular order above, the steps of method 100 may be performed in any order without deviating from the scope of the present disclosure.

In some embodiments, design 302 may be printed with a pigment-based ink and/or a dye-based ink. Designs 302 printed with the pigment-based ink or pigment-based ink and dye-based ink may be more colorfast than designs printed with the dye-based ink alone.

In some embodiments, the adhesive 502 may be a tooth bonding agent and/or a resin tooth bonding agent. In some embodiments, the one or more colorants or pigments 504 may include at least one of pearlescent mica, matte mica, titanium dioxide, zinc oxide, iron oxide, aluminum oxide, or chromium-cobalt-aluminum oxide, Food, Drugs and Cosmetics (FD&C), Drugs & Cosmetics (D&C) color additives, or any other non-toxic pigment or color additive.

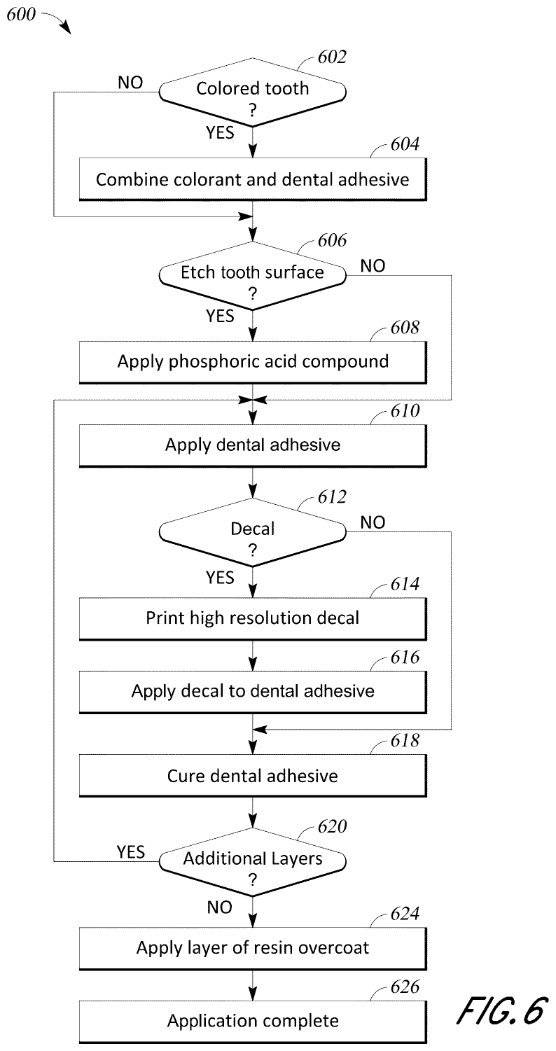

FIG. 6 illustrates a schematic diagram of another method 600 of applying an esthetic alteration to the surface of a patient's tooth. Common features between the method 100 and the method 600 will not be described again but are incorporated here in their entirety. In some embodiments, the step generally indicated by numeral 624 calls for applying a layer of resin overcoat on a top layer of adhesive. The layer of resin overcoat may be a composite resin. In some embodiments, the composite resin may be a translucent or clear flowable composite resin.

In some embodiments, the kit 500 may be a home kit 700 for self-application by a user. In these embodiments, any of the steps of method 100 and/or method 600 may be performed by the user or a dentist or third party. The kit 700 may comprise the sheet of decals 300, a dental adhesive 702, an applicator 704, an adhesive curing device 706, one or more tooth isolators 708, and/or any other items commonly used for application of a dental adhesive. In some embodiments, the kit 700 can include one or more colorants or pigments 710. The user may use the one or more colorants or pigments 710 to color the dental adhesive 702 at step 104 of method 100 and/or step 610 of method 600 by combining the one or more colorants or pigments 710. The one or more colorants or pigments may include at least one of pearlescent mica, matte mica, titanium dioxide, zinc oxide, iron oxide, aluminum oxide, or chromium-cobalt-aluminum oxide, Food, Drugs and Cosmetics (FD&C), Drugs & Cosmetics (D&C) color additives, or any other non-toxic pigment or color additive. In some embodiments the sheet of decals 300 may be delivered to the user with designs 302 already printed on the paper substrate 304. In some embodiments, the dental adhesive 702 may be any commonly used dental adhesive or bonding agent. In some embodiments, the dental adhesive 702 may include eighth-generation dental adhesive. In some embodiments, the dental adhesive 702 may be colored or uncolored. In some embodiments, the dental adhesive 702 may be a unit dose of a resin tooth bonding agent. The applicator 704 may be a brush, such as a microbrush, a cotton ball, cotton swab, roll of cotton, a syringe, and/or any other applicator commonly used for applying dental adhesive to the surface of a tooth. The adhesive curing device 706 may be a dental adhesive light curing unit (LCU). The curing light may be a light emitting diode (LED) LCU, an ultraviolet (UV) LCU, a quartz-tungsten-halogen (QTH) LCU, or any other curing light commonly used for curing dental adhesives. The one or more tooth isolators 708 may be one or more cotton rolls, a dry angle, a dental dam, or any other commonly used tooth isolators.

In some embodiments, the user may use the applicator 704 to apply the dental adhesive 702 to the surface of a tooth at step 110 of method 100 and/or step 610 of method 600. In some embodiments, before the user applies the dental adhesive 702 to the surface of the tooth, the user may use the one or more tooth isolators 708 to isolate the tooth from saliva, bacteria, or any other contaminant. In some embodiments, the user may use the adhesive curing device 706 to cure the at step 118 of method 100 and/or step 618 of method 600.

In some embodiments, the esthetic alteration may be removed by the user with a fingernail of the user, a toothpick, or any other hard object that will not damage the surface of the tooth.

### A. Summary

Although certain aspects, advantages, and features are described herein, it is not necessary that any particular embodiment include or achieve any or all of those aspects, advantages, and features. For example, some embodiments may not achieve the advantages described herein, but may achieve other advantages instead. Any structure, feature, or step in any embodiment can be used in place of, or in addition to, any structure, feature, or step in any other embodiment, or omitted. This disclosure contemplates all combinations of features from the various disclosed embodiments. No feature, structure, or step is essential or indispensable. In addition, although this disclosure describes certain embodiments and examples of tooth alteration systems and methods, many aspects of the above-described systems and methods may be combined differently and/or modified to form still further embodiments or acceptable examples. All such modifications and variations are intended to be included herein within the scope of this disclosure.

Also, although there may be some embodiments within the scope of this disclosure that are not expressly recited above or elsewhere herein, this disclosure contemplates and includes all embodiments within the scope of what this disclosure shows and describes. Further, this disclosure contemplates and includes embodiments comprising any combination of any structure, material, step, or other feature disclosed anywhere herein with any other structure, material, step, or other feature disclosed anywhere herein.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Some embodiments have been described in connection with the accompanying drawings. The figures are drawn to scale, but such scale should not be interpreted to be limiting. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Also, any methods described herein may be practiced using any device suitable for performing the recited steps.

Moreover, while components and operations may be depicted in the drawings or described in the specification in a particular arrangement or order, such components and operations need not be arranged and performed in the particular arrangement and order shown, nor in sequential order, nor include all of the components and operations, to achieve desirable results. Other components and operations that are not depicted or described can be incorporated in the embodiments and examples. For example, one or more additional operations can be performed before, after, simultaneously, or between any of the described operations. Further, the operations may be rearranged or reordered in other implementations. Also, the separation of various system components in the implementations described above should not be understood as requiring such separation in all implementations, and it should be understood that the described components and systems can generally be integrated together in a single product or packaged into multiple products.

In summary, various illustrative embodiments and examples of tooth alteration systems and methods have been disclosed. Although the systems and methods have been disclosed in the context of those embodiments and examples, this disclosure extends beyond the specifically disclosed embodiments to other alternative embodiments and/or other uses of the embodiments, as well as to certain modifications and equivalents thereof. This disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims that follow as well as their full scope of equivalents.

1. A method of applying an esthetic alteration on a tooth surface comprising the steps of:
applying a first layer of dental adhesive to the tooth surface;
placing a decal on the first layer of dental adhesive;
applying at least one additional layer of dental adhesive on top of the decal; and
curing the at least one additional layer of dental adhesive.

2. The method of claim 1, further including the step of curing the first layer of dental adhesive after applying the first layer of dental adhesive.

3. The method of claim 1, wherein the first layer of dental adhesive is a colored dental adhesive comprising a pigment and/or a color additive.

4. The method of claim 3, wherein the pigment comprises at least one of pearlescent mica, matte mica, titanium dioxide, zinc oxide, iron oxide, aluminum oxide, or chromium-cobalt-aluminum oxide, and wherein the color additive comprises Food, Drugs, and Cosmetics (FD&C) and/or Drugs & Cosmetics (D&C) color additives.

5. The method of claim 1, wherein each layer of dental adhesive is a resin tooth bonding agent.

6. The method of claim 1, wherein the tooth surface is etched with a phosphoric acid compound.

7. The method of claim 1, wherein the decal comprises a design printed on a paper substrate.

8. The method of claim 7, wherein the paper substrate comprises rice paper or tissue paper, and the paper substrate comprises a thickness between 8 microns and 10 microns.

9. (canceled)

10. The method of claim 1, further including the step of removing, by a dental professional, a user, or a third party, the esthetic alteration.

11. The method of claim 10, wherein the esthetic alteration is removable with a high-speed dental handpiece and/or a low-speed dental handpiece.

12. (canceled)

13. (canceled)

14. (canceled)

15. (canceled)

16. (canceled)

17. A method of constructing an esthetic alteration kit for a tooth comprising the steps of:
combining a dental adhesive with a pigment to form a colored dental adhesive; and
printing a design on a paper substrate to form a decal.

18. The method of claim 17, wherein the paper substrate comprises rice paper or tissue paper.

19. The method of claim 18, wherein the paper substrate comprises a thickness between 8 microns and 10 microns.

20. The method of claim 17, wherein the design comprises a minimum resolution of 4800×1200 dots per inch.

21. An esthetic alteration kit for a tooth comprising:
a dental adhesive;
an applicator;
an adhesive curing device;
a tooth isolator; and
at least one high resolution decal printed on a paper substrate.

22. The esthetic alteration kit of claim 21, wherein the dental adhesive is a unit dose of resin tooth bonding agent.

23. The esthetic alteration kit of claim 21, wherein the dental adhesive is a colored dental adhesive.

24. The esthetic alteration kit of claim 21, further comprising one or more pigments and/or color additives, wherein the one or more pigments comprise at least one of, pearlescent mica, matte mica, titanium dioxide, zinc oxide, iron oxide, aluminum oxide, or chromium-cobalt-aluminum oxide, and wherein the one or more color additives comprises Food, Drugs, and Cosmetics (FD&C) and/or Drugs & Cosmetics (D&C) color additives.

25. (canceled)

26. (canceled)

27. The esthetic alteration kit of claim 21, wherein the adhesive curing device is a curing light.

28. (canceled)

29. The method of claim 1, further including the step of applying a resin overcoat on the at least one additional layer of dental adhesive.

30. (canceled)

31. The method of claim 7, wherein the decal includes a pigment based ink, and/or a dye based ink.

32. (canceled)

33. The method of claim 17, wherein combining the dental adhesive with the pigment is performed by a dentist, a user, or a third party.

34. The method of claim 1, wherein applying the esthetic alteration is performed by a dentist, a user, or a third party.

35. The method of claim 1, further including the step of removing, by a user, the esthetic alteration with a fingernail, a toothpick, or a hard object that will not damage the surface of the tooth.

36. The esthetic alteration kit of claim 24, wherein dentist, a user, or a third party combines the one or more pigments and/or color additives with the dental adhesive to color the dental adhesive.
